(19) United States
(12) Patent Application Publication
(10) Pub. No.: US 2006/0293255 A1
(43) Pub. Date: Dec. 28, 2006

(54) METHOD FOR REGULATING NUTRIENT ABSORPTION WITH GINSENOSIDES

(75) Inventors: Hang-Ching LIN, Taipei (TW); Wen-Liang CHANG, Taipei (TW); Tsu-Chung CHANG, Taipei (TW); Hsiou-Yu DING, Tainan (TW); Tian Shung WU, Tainan (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: NULIV SCIENCE INC. - A TAIWAN, R.O.C. CORPORATION, Taipei (TW)

(21) Appl. No.: 11/426,064

(22) Filed: Jun. 23, 2006

Related U.S. Application Data
(60) Provisional application No. 60/694,097, filed on Jun. 23, 2005.

(51) Int. Cl. A61K 31/704

(52) U.S. Cl. 514/26

(57) ABSTRACT

There is provided a method for regulating the absorption of a nutrient in a subject, including administering to the subject an effective amount of a ginsenoside compound purified from Panax notoginseng for modulating transportation of the nutrient across gut cells of the subject.

### CROSS-REFERENCE TO RELATED APPLICATION

This application is entitled to and claims the benefit of the priority pursuant to 35 U.S.C. § 119(e) of U.S. Provisional Patent Application No. 60/694,097, filed Jun. 23, 2005, the disclosure of which is hereby incorporated herein by reference.

### BACKGROUND OF THE INVENTION

The present invention relates generally to a method for regulating nutrient absorption, and particularly to a method for regulating nutrient absorption with ginsenosides.

From the study of the human digestive system, it has been found that a huge variety of nutritional substances are obtained by breaking down and digesting the food in the gastrointestinal tract. The gastrointestinal tract is an important route by which the food is digested and absorbed. With regard to absorption, the nutritional substances, such as glucose, amino acids, vitamins and other smaller molecules are absorbed along the entire tract, either by diffusion or by specialized transport processes. Instead of moving freely across the intestinal membrane to the blood stream or lymph, most of these nutritional substances are transported by a tightly regulated mechanism. Based on current understanding in cell biology and physiology, the nutritional substances are transported across the cells with specific transport proteins and channels anchored on the cell membrane.

In the example of glucose transportation, almost all of the cells have a carrier-mediated mechanism for the transport of glucose from blood. For most cells, this transport occurs by facilitated diffusion using one or more of the glucose transporters (GLUT) in a family of facilitated glucose transporters. In these cases, net glucose transport occurs as a result of an inwardly directed chemical gradient for glucose. In a few cell types (e.g. those of intestinal mucosa and renal proximal tubule), uptake of glucose from an extracellular solution can occur against a gradient of glucose in a so-called active transport mechanism, thereby permitting net absorption of glucose from a tissue compartment whose glucose concentration may be lower than that of the blood. There are two ways in which a flow of energy can be coupled to transporters. The primary active transport requires energy be provided by adenosine triphophatase (ATPase). The secondary active transport provides energy from the flow of ions from an area of higher concentration to one of lower concentration.

According to the secondary active transport model described above, Na⁺ binds to transport protein on the luminal side of the cell causing conformational change of the transport protein, which opens the binding site for glucose. Then, glucose binds to the transport protein. The transport protein that is bound with both Na⁺ and glucose is subjected to further conformational change to allow entry of glucose and Na⁺ into the cells. This active transport of glucose involves a direct physical coupling of flows of Na⁺ and glucose, with the energy of the process being derived from the inwardly directed gradient for Na⁺. Since the transport event includes a net movement of charge (the cationic Na⁺ ion with the non-electrolyte glucose), the driving force for this uptake includes both the chemical gradient for Na⁺ and the potential difference across the membrane. As the glucose gradually accumulates in the cell, it is subsequently transported out to the blood vessel via a glucose concentration gradient by facilitated diffusion. Similarly, other nutritional substances may be absorbed with the transport mechanism described above.

Panax notoginseng has been used as a traditional Chinese medicine that mainly serves to invigorate the function of the spleen and increase stamina and endurance. According to current knowledge of Chinese medicine, Panax notoginsenoside extracted from Panax notoginseng can help cerebral blood vessel dilation, increase cerebral blood flow, reduce the oxygen consumption of organism, increase the organism's resistance to oxygen shortage, decrease cerebrovascular resistance, enhance immune function of the organism, prevent shock caused by bleeding, and provide functions of resisting thrombus, blood coagulation, and atherosclerosis.

However, Panax notoginseng has not been implied in regulating nutrient absorption and transportation. None of the study or research has focused on regulating the nutrient absorption using saponin compounds purified from Chinese herbal medicines, particularly Panax notoginseng.

### BRIEF SUMMARY OF THE INVENTION

The present invention provides a method for regulating the absorption of a nutrient in a subject, comprising administering to the subject an effective amount of a ginsenoside compound purified from Panax notoginseng for modulating transportation of the nutrient across gut cells of the subject.

In accordance with an embodiment of the present invention, there is provided a method for enhancing the absorption of a nutrient in a subject, comprising the step of administering to the subject an effective amount of a ginsenoside compound purified from Panax notoginseng for facilitating transportation of the nutrient across gut cells of the subject.

Also in accordance with the present invention, there is provided a method for inhibiting the absorption of a nutrient in a subject, comprising the step of administering to the subject an effective amount of a ginsenoside compound purified from Panax notoginseng for moderating transportation of the nutrient across gut cells of the subject.

According to the invention, the ginsenoside compound is a dammarane compound of Formula (A): 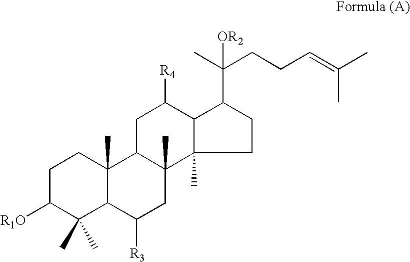
wherein R₁ is selected from the group consisting of H, acetyl, glucopyranosyl, glucopyranosyl-(2-1)-β-D-glucopyranosyl, glucopyranosyl-(2-1)-β-D-xylopyranosyl and glucopyranosyl-(2-1)-β-D-glucopyranosyl-(6-1)-xylopyranosyl; R₂ is selected from the group consisting of H, acetyl, glucopyranosyl, glucopyranosyl-(6-1)-β-D-glucopyranosyl, glucopyranosyl-(6-1)-β-D-xylopyranosyl, glucopyranosyl-(6-1)-α-L-arabinopyranosyl and glucopyranosyl-(6-1)-α-L-arabinofuranosyl; R₃ is selected from the group consisting of H, hydroxy, O-acetyl, O-β-D-glucopyranosyl, O-β-D-glucopyranosyl-(2-1)-β-D-glucopyranosyl, O-β-D-glucopyranosyl-(2-1)-β-D-xylopyranosyl and O-β-D-glucopyranosyl-(2-1)-α-L-rhamnopyranosyl; and R4 is selected from the group consisting of H, hydroxyl and O-acetyl.

According to the invention, the ginsenoside compound preferably is selected from the group consisting of ginsenoside Rb₁ of Formula I: 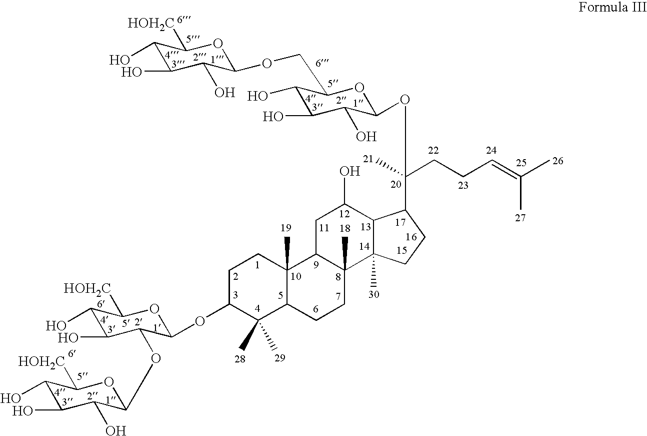
ginsenoside Rg₁ of Formula II: 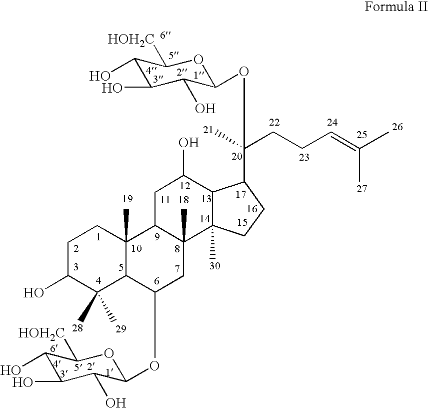
ginsenoside Rh₁ of Formula III: 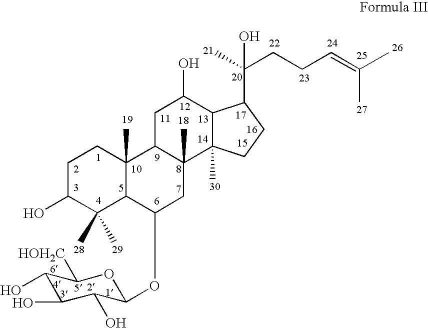
notoginsenoside R₁ of Formula V: 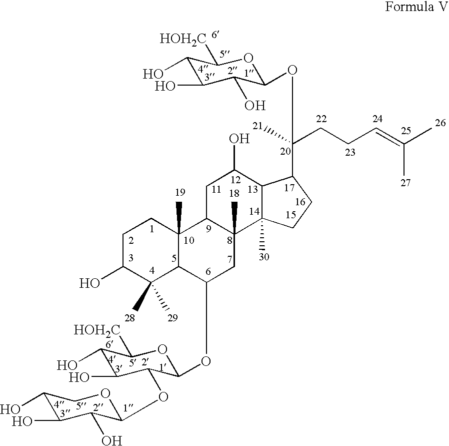
compound K of Formula VII: 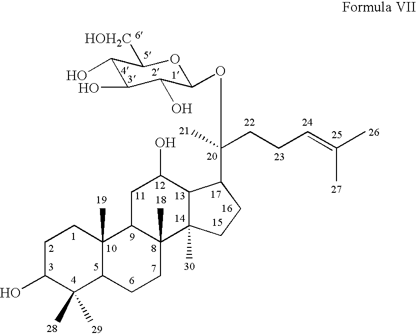
ginsenoside F₁ of Formula VIII: 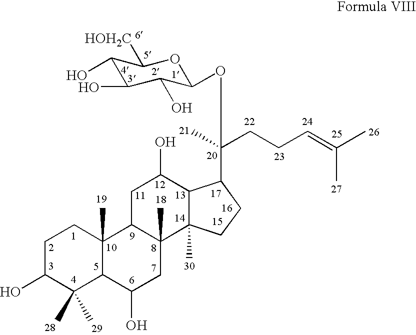
and 20(S)-protopanaxatriol of Formula IX:

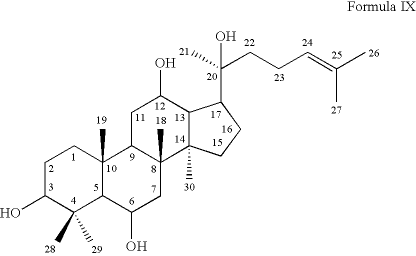

In accordance with preferred embodiments of the invention, the nutrient includes glucose, an amino acid or vitamin. Particularly, the amino acid preferably includes arginine or tryptophan. The vitamin preferably includes folate, among others.

Additional features and advantages of the present invention will be set forth in part in the description which follows, and in part will be apparent from the description, or may be learned by practice of the invention. The features and advantages of the invention will be realized and attained by means of the elements and combinations as described.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments, which are presently preferred. It should be understood, however, that the invention is not limited to the precise arrangements and instrumentalities shown.

In the drawings:

FIG. 1 is a line graph showing the glucose absorption rates measured in the Sink-transport across to basolateral chambers when the Caco2 monolayers were treated with the purified ginsenoside Rb1 of Formula I of selected concentrations;

FIG. 2 is a line graph showing the glucose absorption rates measured in the Sink-transport across to basolateral chambers when the Caco2 monolayers were treated with the purified ginsenoside Rg₁ of Formula II of selected concentrations;

FIG. 3 is a line graph showing the arginine absorption rates measured in the Sink-transport across to basolateral chambers when the Caco2 monolayers were treated with the purified ginsenoside Rg₁ of Formula II of selected concentrations;

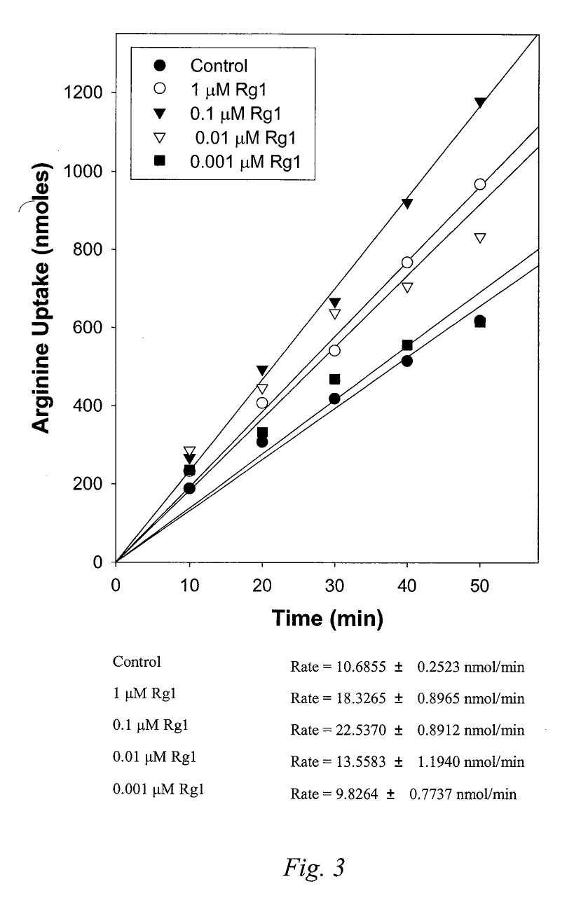

FIG. 4 is a line graph showing the tryptophan absorption rates measured in the Sink-transport across to basolateral chambers when the Caco2 monolayers were treated with the purified ginsenoside, compound K of Formula VII, of selected concentrations; and

FIG. 5 is a line graph showing the folate uptake rates of the Caco2 cells treated with the purified ginsenoside, compound K of Formula VII, of selected concentrations.

### DETAILED DESCRIPTION OF THE INVENTION

To better understand the present invention, the terms used herein are explained in further detail. By medical dictionary definition, a ginsenoside is any of various plant glucosides that form soapy lathers when mixed and agitated with water, used in detergents, foaming agents, and emulsifiers. A ginsenoside as used herein is defined as a triterpenoid saponin compound extracted from ginseng root.

As used herein, the singular forms “a”, “an”, and “the” include plural referents unless the context clearly dictates otherwise. Thus, for example, reference to “a compound” includes a plurality of such compounds.

The term “absorption” as used herein refers to uptake of a nutrient via a passage through the intestinal epithelium and into the blood or lymph.

The term “purified” as used herein refers to a chemical process by which pure compounds or substances of at least about 90%, preferably up to 100%, by weight purity are isolated from a crude or natural form.

The term “gut cells” as used herein generally include enterocytes, mucosal cell, and cells of intestinal epithelium responsible for nutrient absorption of the body.

The term “subject” as used herein refers to any animal, preferably including humans, where absorption of nutrients occurs across gut cells in the subject's gastrointestinal tract.

The present invention provides a method for regulating the absorption of a nutrient in a subject, comprising administering to the subject an effective amount of a ginsenoside compound purified from Panax notoginseng for modulating transportation of the nutrient across gut cells of the subject. The method comprises administering an effective amount of the ginsenoside compound to the subject for regulating nutrient transportation across a monolayer of the gut cells lining a gastrointestinal tract, so that the absorption of the nutrient is regulated in a subject. The effective amount of the ginsenoside compound administered to the subject is the concentration which maintains viability in the gut cells and enhances or inhibits the nutrient transportation across cell membranes of the gut cells, and is preferably about 0.001 μM to about 5 μM. Since the ginsenoside compounds purified from Panax notoginseng may enhance or inhibit the transportation of a nutrient across the monolayer of the gut cells, the absorption of the nutrient is regulated to maintain a desired level of the absorption of the subject, depending on the ginsenoside compounds administered. The ginsenoside compounds may be formulated into tablets, pills, capsules, liquid formulations and powder to be orally administered to the subject suffering from a nutrient absorption problem. Also, the ginsenoside compounds may be optionally mixed with other nutrient factors, additives, stabilizing agents, carriers, binders and fillers to produce dietary supplements, beverages, and food for anyone in need of regulated nutrient absorption. It may be apparent to one skilled in the art in view of the present disclosure to administer the ginsenoside compounds in combination or in a cocktail manner with other ginsenosides and astragalosides to provide a synergistic or accumulative effect on the nutrient absorption. In addition, the purified ginsenoside compounds may also be purified from other Chinese herbal plants or vegetation to provide the same regulatory effect on nutrient absorption function.

The ginsenoside compounds may be prepared by any standard methodology or known methods or knowledge in the art. According to the invention, the ginsenoside compounds purified from Panax notoginseng include the ginsenosides. They may be purified by other available extraction and isolation methods known to those skilled in the art.

According to the invention, the ginsenoside compound is a dammarane compound of Formula (A): 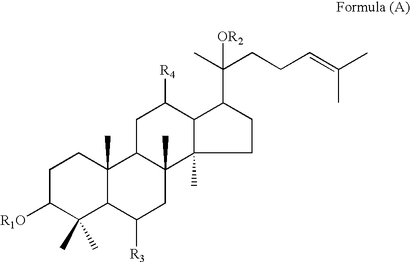
wherein R₁ is selected from the group consisting of H, acetyl, glucopyranosyl, glucopyranosyl-(2-1)-β-D-glucopyranosyl, glucopyranosyl-(2-1)-β-D-xylopyranosyl and glucopyranosyl-(2-1)-β-D-glucopyranosyl-(6-1)-xylopyranosyl; R₂ is selected from the group consisting of H, acetyl, glucopyranosyl, glucopyranosyl-(6-1)-β-D-glucopyranosyl, glucopyranosyl-(6-1)-β-D-xylopyranosyl, glucopyranosyl-(6-1)-α-L-arabinopyranosyl and glucopyranosyl-(6-1)-α-L-arabinofuranosyl; R₃ is selected from the group consisting of H, hydroxy, O-acetyl, O-β-D-glucopyranosyl, O-β-D-glucopyranosyl-(2-1)-β-D-glucopyranosyl, O-β-D-glucopyranosyl-(2-1)-β-D-xylopyranosyl and O-β-D-glucopyranosyl-(2-1)-α-L-rhamnopyranosyl; and R₄ is selected from the group consisting of H, hydroxyl and O-acetyl.

According to an embodiment of the invention, the ginsenoside compounds may be obtained by a method comprising the steps of grinding the root of the Panax notoginseng; extracting the ground materials with alcohol to produce an alcohol extract; separating the alcohol extract of the root of Panax notoginseng and purifying the alcohol extract to give five known ginsenosides including:
ginsenoside Rb₁ of Formula I (hereinafter “Rb₁”): 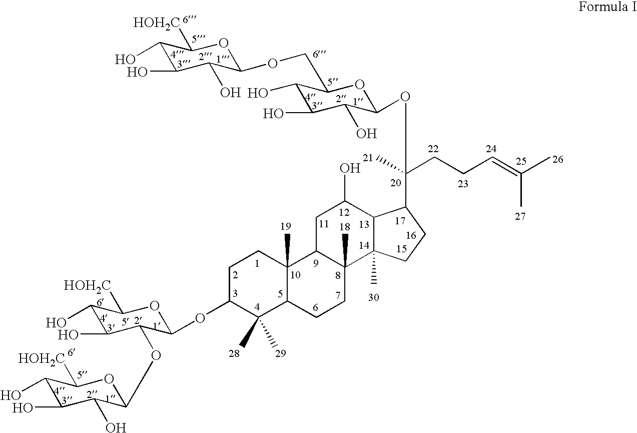
ginsenoside Rg₁ of Formula II (hereinafter “Rg₁”): 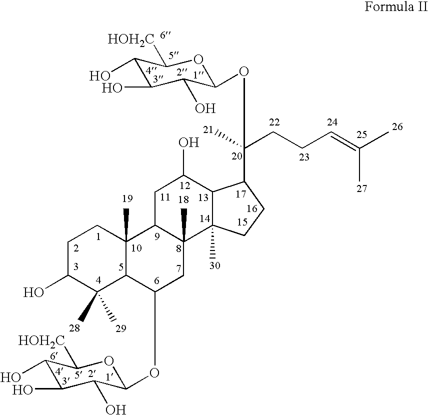
ginsenoside Rh₁ of Formula III (hereinafter “Rh₁”): 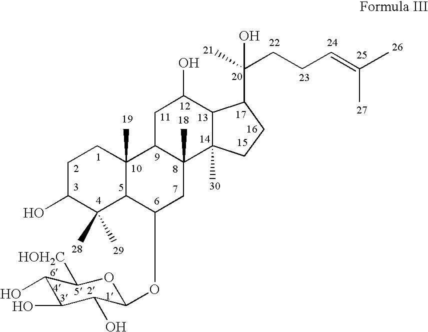
ginsenoside Re of Formula IV (hereinafter “Re”): 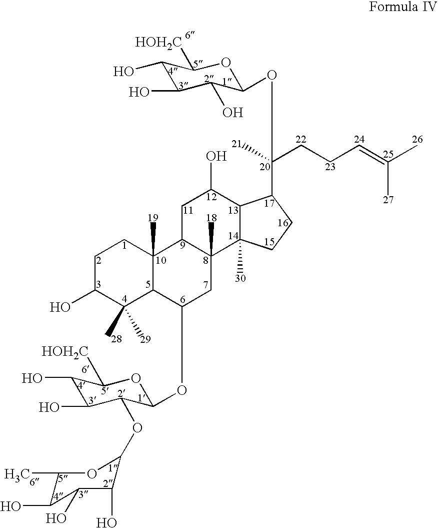
and
notoginsenoside R₁ of Formula V (hereinafter “R₁”):

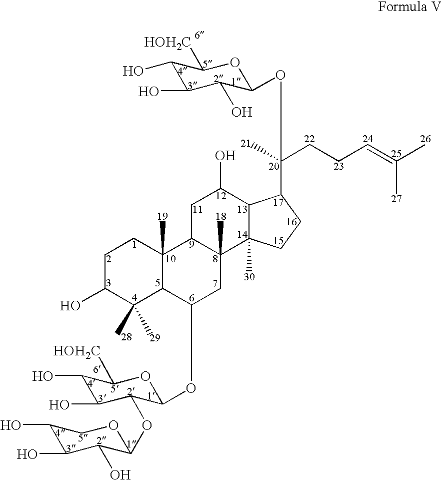

According to the embodiments of the invention, the alcohol extract of Panax notoginseng may be separated and purified with absorbent resin, silica gel and reversed phase chromatography. Then, the ginsenosides Rb₁ and Rg₁ may further be hydrolyzed by naringinase to yield metabolites including:
ginsenoside F₂ of Formula VI (hereinafter “GF2”): 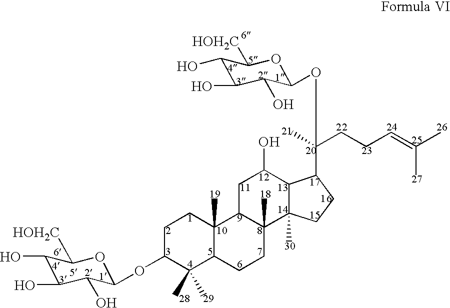
compound K of Formula VII (hereinafter “CK”): 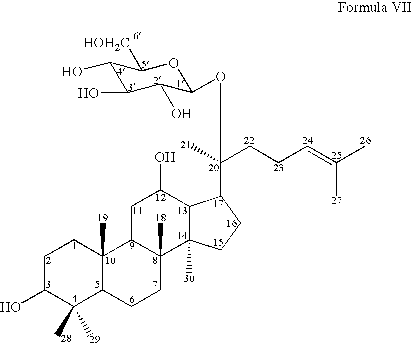
ginsenoside F₁ of Formula VIII (hereinafter “GF1”): 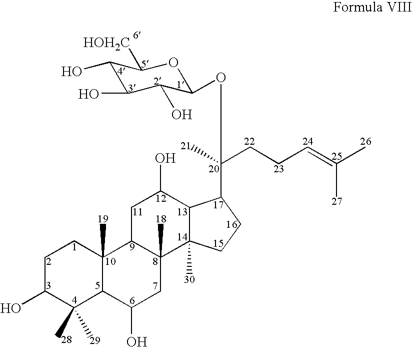
and
20(S)-protopanaxatriol of Formula IX (hereinafter “Rga”).

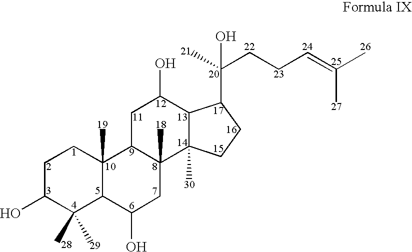

Since the ginsenoside compounds purified from Panax notoginseng can enhance or inhibit transportation of nutrient across the cell membranes of the gut cells, the nutrient absorption is regulated to maintain the desired nutrient level in a subject, depending on the groups of the ginsenoside compounds administered. The ginsenoside compounds may be formulated into tablets, pills, capsules, liquid formulations and powder to be orally administered in the individual with a nutrient absorption problem or mal-absorption syndrome, which is an alteration in the ability of the intestine to absorb nutrients adequately into the bloodstream. For example, in an embodiment of the preparation of the liquid formulation, one or more of the ginsenoside compounds may be dissolved in any solvent, preferably in a co-solvent, to produce a liquid formulation of the ginsenoside compounds (such as, 10 mg of any of the ginsenoside compounds may be dissolved in one mL of Transcutol® P [2-(2-ethoxyethoxy)ethanol]). Also, the ginsenoside compounds may be optionally mixed with other nutrient factors, additives, stabilizing agents, carriers, binders and fillers to produce dietary supplements, beverages, food and animal feeds.

The invention provides a method for enhancing the absorption of a nutrient in a subject, comprising the step of administering an effective amount of a ginsenoside compound purified from Panax notoginseng for facilitating transportation of the nutrient across the gut cells of the subject. The nutrient preferably includes glucose, an amino acid or vitamin; wherein the amino acid preferably includes arginine or tryptophan; and the vitamin preferably includes folate, among others.

According to an embodiment of the invention, the absorption of glucose was enhanced by facilitating the transportation of glucose across the gut cells of the subject with administration of the ginsenoside compound at a concentration of about 0.001 μM to about 5 μM; wherein the ginsenoside compound includes Rb₁ of Formula I, CK of Formula VII, GF1 of Formula VIII or Rga of Formula IX.

According to an embodiment of the invention, the absorption of arginine was enhanced by facilitating the transportation of arginine across the gut cells of the subject with administration of the ginsenoside compound at a concentration of about 0.001 μM to about 5 μM; wherein the ginsenoside compound includes Rb₁ of Formula I, Rg₁ of Formula II, CK of Formula VII, Rh₁ of Formula III, GF1 of Formula VIII or Rga of Formula IX.

According to an embodiment of the invention, the absorption of tryptophan was enhanced by facilitating transportation of tryptophan across the gut cells of the subject with administration of the ginsenoside compound at a concentration of about 0.001 μM to about 5 μM; wherein the ginsenoside compound includes CK of Formula VII or Rg₁ of Formula II.

According to an embodiment of the invention, the absorption of folate was enhanced by facilitating transportation of folate across the gut cells of the subject with administration of the ginsenoside compound at a concentration of about 0.001 μM to about 5 μM; wherein the ginsenoside compound includes CK of Formula VII or Rb₁ of Formula I.

The invention also provides a method for inhibiting the absorption of a nutrient in a subject, comprising the step of administering an effective amount of a ginsenoside compound purified from Panax notoginseng for moderating transportation of the nutrient across the gut cells of the subject. The nutrient preferably includes glucose or vitamin; wherein the vitamin preferably includes folate, among others.

According to the invention, the absorption of glucose was inhibited by moderating the transportation of glucose across the gut cells of the subject with administration of the ginsenoside compound at a concentration from 0.001 to 5 μM; wherein the ginsenoside compound includes Rg₁ of Formula II or Rh₁ of Formula III.

According to an embodiment of the invention, the absorption of folate is inhibited by moderating the transportation of folate across the gut cells of the subject with administration of the ginsenoside compound at a concentration from 0.001 to 5 μM; wherein the ginsenoside compound includes Rg₁ of Formula II or Rh₁ of Formula III.

The present invention is more specifically explained by the following examples. However, it should be noted that the present invention is not limited to these examples in any manner.

### EXAMPLE 1

### Regulatory Effects of Purified Ginsenosides on Glucose Absorption

Cell Culture

To evaluate the effect of the purified ginsenoside compound on the uptake of nutrient substances across the intestinal lumen, Caco-2 cells were grown on permeable filter as an experimental model. Caco2 cells originate from human colonic adenocarcinoma and spontaneously differentiate into an enterocyte-like phenotype after two weeks. The Caco-2 cell line, derived from a human colorectal carcinoma, has been used as an in vitro model system for studying drug absorption in the gastrointestinal tract. These cells form monolayers with well-developed tight-junctions, and have been evaluated in details as an in vitro model to study both transcellular transport of nutrients and drugs in intestinal lumen.

Caco-2 cells were obtained from the ATCC (American Type Culture Collection). The cells were maintained in Dulbecco's modified Eagle medium (DMEM) containing 4.5 g/L glucose and 25 mM Hepes, supplemented with 10% fetal calf serum, 100 U/mL penicillin G and 10 μg/L streptomycin. The medium was changed every second day. The cells were routinely checked for Mycoplasma in monthly intervals. Caco-2 cells were cultured on semi-permeable membranes to differentiate into a highly functionalized epithelial barrier with remarkable morphological and biochemical similarity to the small intestinal columnar epithelium. The Caco-2 cell monolayers could therefore be used to study the membrane transport properties of many compounds. To trypsinize the cells, the culture dish was washed once with phosphate-buffered saline (PBS) followed by adding trypsine-EDTA for 10 minutes. The trypsinized cells were separated and filtered into single cells using a 35 μm strainer cap (Falcon 2235) before being seeded for further experiments.

Cell Viability Assay

In order to investigate whether the purified ginsenosides were toxic to the Caco2 cells, a cell viability assay was carried out using culture medium supplemented with 1% and 10% FBS, respectively. The cells were seeded at a concentration of 5000 cells/well in a 96-well plate. To eliminate the boundary effect of the cell growth, the cells were only seeded in 60 wells of the middle area of the plate, whereas 36 wells at the surrounding area of the plate were filled only with 100 μL of PBS. Once the cells were attached to the plate, the cells were incubated in medium containing the purified ginsenosides at various concentrations (0, 0.1, 1, 10 and 50 μM). After 3 days, the culture medium was replaced with fresh medium containing the same compounds and incubated for 2 more days before the cells were tested for cell viability.

The cell viability was determined by a Cell Counting Kit-8 (CCK-8, Dojindo Laboratories, Kumamoto, Japan) assay that was based on redox reaction of NADH in the living cells with cell proliferation reagent WST-8. WST-8 was reduced by dehydrogenases in electron transport chain (ETC) of mitochondria in the cells to give a yellow-colored formazan product, which was soluble in the tissue culture medium. The amount of formazan dye generated by the activity of dehydrogenases in the cells was directly proportional to the number of the living cells. Therefore, a greater light absorbance detected by ELISA reader at wavelength of 450 nm indicated presence of a larger number of the living cells.

The CCK-8 assay was carried out by adding 10 μL of the CCK-8 reagent in each well of a 96-format plate. The plate was then covered with aluminum foil and further incubated for two hours before measuring for absorbance at wavelengths of 450 nm using an ELISA reader.

Glucose Uptake Assay

Caco-2 cells (5×10⁴) were seeded in a 48-well plate and maintained in culture medium (DMEM with 10% FBS, 1% nonessential amino acids, L-glutamine, penicillin G (100 U/mL), streptomycin (10 μg/mL), and amphotericin B (2.5 μg/mL) in a 37° C. incubator for 10 days for the cells to differentiate. The culture medium was changed once every two days. The cells were then washed with PBS before replenishing with the culture medium containing 5% FBS and the purified ginsenosides at the various concentrations (0.01, 0.1 and 1 μM) for 48 hours. The Caco2 cells were washed out of remaining glucose with PBS and replaced in the glucose buffer (80 mM NaCl, 100 mM mannitol, 20 mM Tris-HCl, pH 7.4, 3 mM K₂HPO₄, 1 mM CaCl₂, 1 mg/mL BSA) for 1 hour. Glucose uptake was initiated by replacing the glucose buffer with 0.2 ml of glucose buffer containing 2 μCi/mL of ¹⁴C-glucose and unlabeled cold glucose to give a final glucose concentration of 25 mM. Glucose uptake was stopped by removing the glucose buffer and washing with PBS at designated time intervals. The cells were lysed in 0.2 mL of 0.2 N NaOH, and 20 μL of the cell lysate were transferred to the filter-bottomed UniFilter plates (Perkin-Elmer, Wellesley, Mass., USA) and dried in a vacuum oven at 37° C. The bottom of the UniFilter plate was sealed and 25 μL of the counting solution were added into each well. Adhesive plate sealers were used in place of the lids and radioactivity of each sample was counted using the microplate liquid scintillation counter (TopCount, Packard NXT, Packard BioScience Company, Meriden, Conn., USA). The amount of glucose accumulated in the cells was calculated and normalized to protein concentration, and uptake rate was expressed as nanomoles of glucose per minutes per milligram of cell protein (nmol/min/mg). Protein concentration was determined by a standard Bicinchoninic acid (BCA) protein assay. Nonspecific glucose uptake was measured by the adding 2 μCi of L-[¹⁴C]-glucose and subtracting from each determination to obtain specific glucose uptake.

Glucose Absorption Assay

For glucose absorption assay, 0.3 mL (10⁵ cell/ml) of Caco2 cells were seeded into the apical chamber of each transwell and the basolateral chamber was added with 1 ml of culture medium (as indicated above). Costar transwell inserts (No. 3414, Coming Incorporated, NY, USA) separate each well in 24-well plates into an apical chamber (defined as a chamber above the insert) and a basolateral chamber (defined as a chamber below the insert). The culture medium in each transwell was changed every 2 days in both of the apical and basolateral chambers. To ensure the integrity of tight junction of Caco2 monolayer membrane formed in the transwell, a Trans-Epithelial Electrical Resistance (TEER) assay was conducted using Millicell®-ERS (Millipore EVOM-6; World Precision Instrument, Sarasota, Fla., USA) to measure the TEER between apical and basolateral chambers of the transwell. As the measured TEER reached 300 to 450 Ωcm² with the presence of differentiated brush border on the basolateral side of the cell on the 14th to 21st day of the culture, the Caco2 monolayer was ready for the glucose absorption test. The TEER values were taken before, during, and after each experiment to justify consistency of the data collected.

In the glucose absorption assay, the Caco2 cell monolayer was pretreated with the culture medium containing 5% FBS and purified ginsenosides at various concentrations (1, 0.1, and 0.01 μM) for 2 days before the Caco2 cell monolayer was further cultured in a glucose absorption buffer (80 mM NaCl, 100 mM mannitol, 20 mM Tris-HCl, pH 7.4, 3 mM K₂HPO₄, 1 mM CaCl₂, 1 mg/mL BSA) for 1 hour. Glucose absorption was initiated by replacing the culture medium in the basolateral chamber with fresh glucose absorption buffer and the culture medium in the apical chamber with 0.2 mL of the glucose absorption buffer containing 2 μCi/ml of radioactive D-[¹⁴C]-glucose (60 Ci/mmol, American Radiolabeled Chemicals, ARC, St. Louis, Mo., USA) and unlabeled cold glucose to give glucose at a final concentration of 25 mM. A series of five 10-μL samples were taken from the basolateral chamber at every 5 or 10 minute intervals. In order to maintain the constant buffer volume, same amount of sample buffer were added back to the basolateral chamber after withdrawn of each sample. The samples were transferred to the filter-bottomed UniFilter plates (Perkin-Elmer, Wellesley, Mass., USA) and dried in a vacuum oven at 37° C. The bottom of the UniFilter plate was sealed and 25 μL of the counting solution were added into each well. Adhesive plate sealers were used in place of the lids and radioactivity of each sample was counted using the microplate liquid scintillation counter (TopCount, Packard NXT, Packard BioScience Company, Meriden, Conn., USA). The effect of test compounds on glucose absorption was expressed as the nmoles of glucose accumulated in the basolateral chamber with respect to time in minutes (nmol/min). The glucose absorption rate was obtained by calculating the slope of the straight line from the time zero to the end point of the graphic data of each figure.

In the cell viability test, the purified ginsenosides were not toxic to Caco2 cells at a concentration from 0.1 to 50 μM in the culture medium containing 10% FBS. However, Rg₁ at concentrations of 10 μM and 50 μM exhibited cytotoxicity to Caco2 cells in the culture medium containing 1% FBS. Therefore, the purified ginsenoside was administered in the subsequent experiments at a concentration range with no adverse effect on cell viability and morphology. Preferably, the purified ginsenoside was administered at a concentration range of about 0.01 μM to about 5 μM.

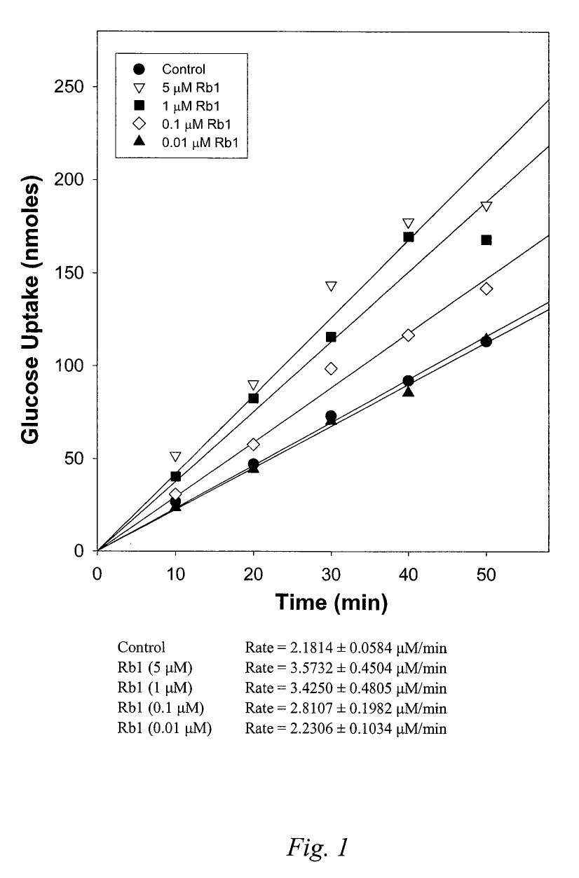

From the glucose absorption assay results shown in Table 1, it was found that a purified ginsenoside selected from Rb₁ of Formula I, CK of Formula VII, Rg₁ of Formula II, Rh₁ of Formula III, GF1 of Formula VIII or Rga of Formula IX, had a regulatory effect on the glucose transport across the Caco2 cell monolayer. That is, the purified ginsenoside either enhances or inhibits the glucose transport across the Caco2 cell monolayer. The glucose transport rate was calculated as a gradient of the curve representing the total amount of glucose measured as gM in the basolateral chamber of the transwell with respect to time in minutes. Referring to FIG. 1, on one hand, glucose transport rate was increased when the Caco2 cell monolayer was treated with Rb₁ of Formula I at a concentration of 0.1 μM to 5 μM.

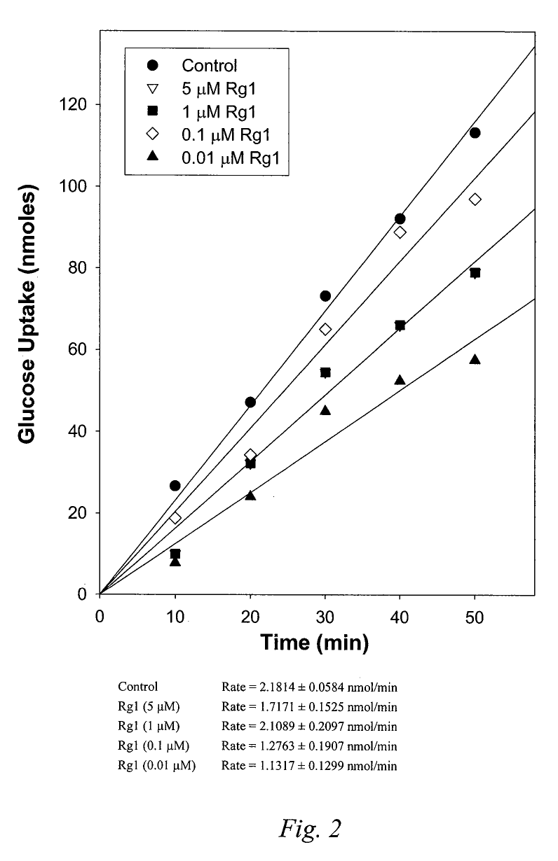

On the other hand, two purified ginsenosides, Rg₁ of Formula II and Rh₁ of Formula III, both show inhibitory effect on glucose absorption as shown in Table 1. Referring to FIG. 2, the glucose transportation was clearly inhibited when the Caco2 cell monolayer was treated with Rg₁ of Formula II at a concentration of 0.01 μM to 1 μM. The regulatory effects of the purified ginsenosides on the glucose transport across the Caco2 cell monolayer are listed in Table 1 below, wherein the arrow that points up represents the enhancing effect on the glucose transport, and the arrow that points down represents the inhibitory effect on the glucose transport.

TABLE 1
Regulatory effects of purified ginsenosides on glucose transport

| | | Transport rate | Percentage | |
|---|---|---|---|---|
| Compound (μM) | | (nmol/min) | (%) | * |
| Control | | 2.1814 ± 0.0584 | 100 | — |
| Rb₁ | 1 | 3.4250 ± 0.4805 | 157.01 | ↑ |
| | 0.1 | 2.8107 ± 0.1982 | 128.85 | ↑ |
| | 0.01 | 2.2306 ± 0.1034 | 102.26 | ↑ |
| CK | 1 | 2.9008 ± 0.2184 | 132.98 | ↑ |
| | 0.1 | 2.8689 ± 0.2783 | 131.52 | ↑ |
| | 0.01 | 3.3164 ± 0.1911 | 152.03 | ↑ |
| Rg₁ | 1 | 2.1089 ± 0.2097 | 96.68 | ↓ |
| | 0.1 | 1.2763 ± 0.1907 | 58.51 | ↓ |
| | 0.01 | 1.1317 ± 0.1299 | 51.88 | ↓ |
| Rh₁ | 1 | 1.3310 ± 0.8356 | 61.02 | ↓ |
| | 0.1 | 1.6329 ± 0.1976 | 74.86 | ↓ |
| | 0.01 | 1.3568 ± 0.1090 | 62.20 | ↓ |
| GF1 | 1 | 3.2862 ± 0.3429 | 150.65 | ↑ |
| | 0.1 | 3.3551 ± 0.3248 | 153.80 | ↑ |
| | 0.01 | 3.0783 ± 0.9550 | 141.12 | ↑ |
| Rga | 1 | 2.2689 ± 0.2598 | 104.01 | ↑ |
| | 0.1 | 3.6462 ± 0.4105 | 167.15 | ↑ |
| | 0.01 | 2.5454 ± 0.7808 | 116.69 | ↑ |

Therefore, it is concluded that the absorption of a glucose can be regulated with the administration of the ginsenoside purified from Panax notoginseng, including Rb₁ of Formula I, Rg₁ of Formula II, CK of Formula VII, Rh₁ of Formula III, GF1 of Formula VIII or Rga of Formula IX.

### EXAMPLE 2

### Regulatory Effect of Purified Ginsenosides on Arginine Absorption

Arginine Absorption Assay

In measuring transport of arginine across the Caco-2 cell monolayer, both sides of the transwells were washed with arginine incubation buffer consisting of: 137 mM NaCl, 10 mM Hepes, 0.3 mMNaH₂PO₄, 0.3 mM K₂HPO₄, 5.4 mM KCl, 2.8 mM CaCl₂, 1 mM MgSO₄, 10 mM glucose, adjusted to pH 7.4. Then, the cell layer was pre-incubated in the incubation buffer at 37° C. for 1 h. The volumes of incubation buffer were 0.2 mL and 0.9 mL in the apical and basolateral chambers, respectively. The cells were replaced with fresh incubation medium in both chambers prior to the transport experiment. The transport experiment was initiated by replacing the incubation solution on the apical side with solution containing 10 mM of L-arginine in which 0.125 μCi/mL of L-[³H]-arginine was included. At designated time intervals, 10 μL-solution samples were removed from the basolateral side and radioactivity of each sample was counted using a microplate liquid scintillation counter (TopCount, Packard NXT). During the experiment, when a 10 μL-solution sample was removed from the basolateral side every time, 10 μL buffer was supplemented to keep the volume constant. The uptake of [³H]-mannitol was used to correct for nonspecific transport of molecules across the monolayer membrane. Results were expressed as the nanomoles of arginine transport across the Caco-2 cell monolayers with respect to time in minutes (nmol/min).

From the results of the arginine absorption assay shown in Table 2, it was found that purified ginsenosides, such as Rb₁ of Formula I, CK of Formula VII, Rg₁ of Formula II, Rh₁ of Formula III, GF1 of Formula VIII and Rga of Formula IX had regulatory effects on the arginine transport across the Caco-2 cell monolayer. The regulatory effects of the purified ginsenosides on the arginine transport in Caco-2 cells are listed in Table 2 below, wherein the arrows that points up represent the enhancing effect on the arginine transport.

TABLE 2
Regulatory effects of purified ginsenosides on Arginine transport

| | | Transport rate | Percentage | |
|---|---|---|---|---|
| Compound (μM) | | (nmol/min) | (%) | * |
| Control | | 10.6855 ± 0.2523 | 100 | — |
| CK | 1 | 14.1530 ± 0.9315 | 132.45 | ↑ |
| | 0.1 | 14.9247 ± 1.4850 | 139.67 | ↑ |
| | 0.01 | 12.8943 ± 0.3197 | 120.67 | ↑ |
| Rb₁ | 1 | 16.1484 ± 0.6228 | 151.12 | ↑ |
| | 0.1 | 11.8699 ± 1.9300 | 111.08 | ↑ |
| | 0.01 | 9.6487 ± 0.9377 | 90.30 | |
| Rh₁ | 1 | 14.3209 ± 0.7418 | 134.02 | ↑ |
| | 0.1 | 11.6615 ± 0.8085 | 109.13 | ↑ |
| | 0.01 | 11.2792 ± 0.7768 | 105.56 | ↑ |
| Rg₁ | 1 | 18.3265 ± 0.8965 | 171.51 | ↑ |
| | 0.1 | 22.5370 ± 0.8912 | 210.91 | ↑ |
| | 0.01 | 13.5583 ± 1.1940 | 126.89 | ↑ |
| GF1 | 1 | 10.3711 ± 0.6574 | 97.06 | |
| | 0.1 | 12.6865 ± 0.2964 | 118.73 | ↑ |
| | 0.01 | 13.5425 ± 1.8630 | 126.74 | ↑ |
| Rga | 1 | 10.1920 ± 1.2390 | 95.38 | |
| | 0.1 | 10.7555 ± 0.4532 | 100.66 | |
| | 0.01 | 12.6265 ± 0.9875 | 118.16 | ↑ |

It is concluded that the absorption of arginine can be regulated with the administration of the ginsenoside purified from Panax notoginseng, including Rb₁ of Formula I, Rg₁ of Formula II, CK of Formula VII, Rh₁ of Formula III, GF1 of Formula VIII or Rga of Formula IX.

### EXAMPLE 3

### Regulatory Effect of Purified Ginsenosides on Tryptophan Absorption

Trytophan Absorption Assay

The experimental procedures similar to those in Example 2 were used for measuring the uptake of tryptophan molecules across the Caco-2 membrane, except using a tryptophan incubation buffer consisting of 137 mM choline chloride, 10 mM Hepes, 0.6 mM KH₂PO₄, 5.4 mM KCl, 2.8 mM CaCl₂, 1 mM MgSO₄, and 10 mM glucose, and having its pH adjusted to 7.4. Results were expressed as the nanomoles of tryptophan transport across the Caco-2 cell monolayers with respect to time in minutes (nmol/min).

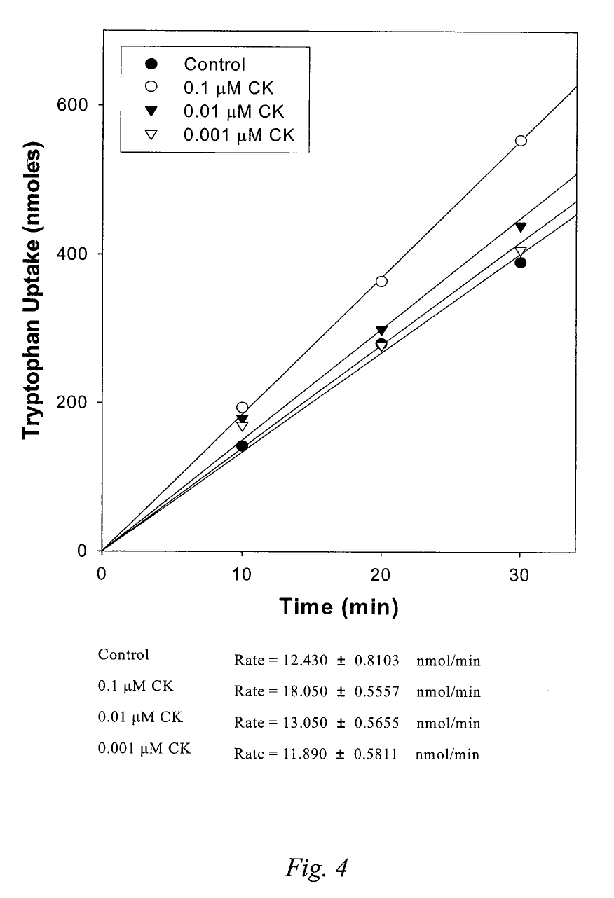

From the tryptophan absorption assay results shown in Table 3, it was found that purified ginsenosides such as CK of Formula VII and Rg₁ of Formula II had regulatory effects on the tryptophan transport across the Caco-2 cell monolayer. That is, the purified ginsenoside could enhance the tryptophan transport across the Caco-2 cell monolayer. Referring to FIG. 4, tryptophan transport rate was increased when the Caco-2 cell monolayer was treated with CK of Formula VII at a concentration from 0.01 to 0.1 μM. As shown in Table 3, the tryptophan transport rate was increased when the Caco-2 cell monolayer was treated with Rg₁ of Formula II at a concentration from 0.001 μM to 0.1 μM. The regulatory effects of the purified ginsenosides on the tryptophan transport across the Caco2 cell monolayer are listed in Table 3 below.

TABLE 3
Regulatory effects of purified ginsenosides
on Tryptophan transport

| | | Transport rate | Percentage | |
|---|---|---|---|---|
| Compound (μM) | | (nmol/min) | (%) | * |
| Control | | 12.430 ± 0.8103 | 100 | — |
| CK | 0.1 | 18.050 ± 0.5557 | 145.21 | ↑ |
| | 0.01 | 13.050 ± 0.5655 | 104.99 | ↑ |
| | 0.1 | 17.710 ± 0.5948 | 142.48 | ↑ |
| Rg₁ | 0.01 | 16.590 ± 1.4190 | 133.47 | ↑ |
| | 0.001 | 16.180 ± 2.8700 | 130.17 | ↑ |

It is concluded that the absorption of tryptophan can be regulated with the administration of the ginsenoside purified from Panax notoginseng, including Rg₁ of Formula II or CK of Formula VII.

### EXAMPLE 4

### Regulatory Effects of Purified Ginsenosides on Folate Uptake

Folate Uptake Assay

The Caco2 cells were subjected to folate uptake test in a manner similar to that described in the glucose uptake assay in Example 1 above. In the folate uptake test, the Caco2 cells were pretreated with the culture medium containing 5% FBS and purified ginsenosides at a concentration of 0.1 μM for 2 days before the cells were cultured in a folate uptake buffer (Hank's balanced salt solution, supplemented with 0.14 g/L CaCl₂, 0.1 g/L MgCl₂, and 0.1 g/L MgSO₄, pH 6.0) for 1 hour. The buffer was then aspirated, and uptake was initiated by adding 0.2 mL of fresh folate uptake buffer containing 2 μCi/mL radioactive folate (3,5,7,9-³H-folic acid, 25 mCi/mmol, ARC) and cold, unlabeled folate giving a final folate concentration of 5 μM. Folate uptake was terminated by removing the uptake buffer at designated time intervals. The cells were then washed three times with ice-cold PBS and lysed by adding 0.2 mL of 0.2 N NaOH, followed by incubating at 65° C. for 20 min. Intracellular uptake of ³H-folate was determined by transferring 20 μL of the cell lysate to the filter-bottomed UniFilter plates (Perkin-Elmer) and counting as described previously in Example 1. The amount of folate accumulated in the cells was calculated and normalized to protein concentration, and uptake rate was expressed as picomoles of folate per minutes per milligram of cell protein (pmol/min/mg). Protein concentration was determined by a standard Bicinchoninic acid (BCA) protein assay as described above.

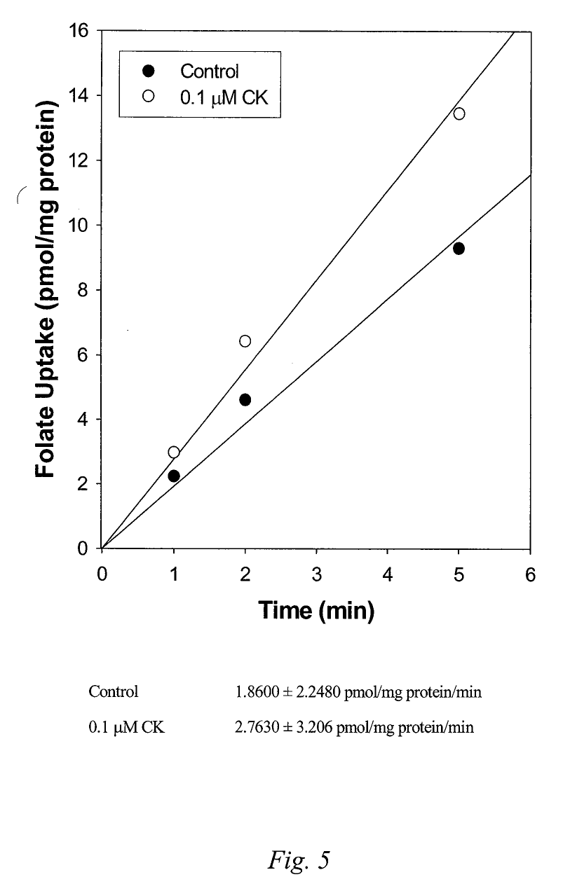

Referring to FIG. 5 and Table 4, Caco2 cells treated with either CK of Formula VII or Rb₁ of Formula I at a concentration of 0.1 μM were found to exhibit an increased folate uptake from the control group having non-treated Caco2 cells. In contrast, the Caco2 cells treated with either Rg₁ of Formula II or Rh₁ of Formula III at a concentration of 0.1 μM had their folate uptakes decreased as shown in Table 4. The regulatory effects of the purified ginsenosides on the folate uptake in Caco2 cells are listed in Table 4 below, wherein the arrows that point up represent the enhancing effect on folate uptake, and the arrows that point down represent the inhibiting effect on folate uptake.

TABLE 4
Regulatory effect of purified ginsenosides on folate uptake

| | Transport rate | Percentage | |
|---|---|---|---|
| Compound (μM) | (pmol/mg/min) | (%) | |
| Control | 1.8600 ± 2.2480 | 100 | — |
| CK 0.1 | 2.7630 ± 3.206 | 148.55 | ↑ |
| Control | 1.7210 ± 0.1611 | 100 | — |
| Rb1 0.1 | 1.8710 ± 0.0320 | 108.72 | ↑ |
| Control | 1.7210 ± 0.1611 | 100 | — |
| Rh1 0.1 | 0.8494 ± 0.453 | 49.36 | ↓ |
| Control | 1.7210 ± 0.1611 | 100 | — |
| Rg1 0.1 | 1.0780 ± 0.1534 | 62.64 | ↓ |

It is concluded that the uptake of folate can be regulated with the administration of the ginsenoside purified from Panax notoginseng, including Rb₁ of Formula I, Rg₁ of Formula II, CK of Formula VII or Rh₁ of Formula III.

Although the above examples described regulating nutrient absorption of the colon cancer cells, it should be noted that the present invention is not limited as such. The gut cells and cells of gastrointestinal system should also be expected to benefit from the regulatory effect of the ginsenoside compounds proposed in the present invention as long as these cells have similar nutrient transporting mechanisms. Besides a regulatory role in glucose and folate absorption, the ginsenoside compounds described in the present invention may equivalently apply to regulate absorption of nutrients which include vitamins, amino acids, hormones, growth factors, and other elements important for cell metabolism. Moreover, the nutrient absorption test and nutrient uptake test described in the embodiments may be implemented interchangeably for assessing and evaluating the regulatory effect of the purified ginsenoside on the nutrient absorption of the individual according to the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method for regulating the absorption of a nutrient in a subject, comprising administering to the subject an effective amount of a ginsenoside compound purified from Panax notoginseng for modulating transportation of the nutrient across gut cells of the subject.

2. The method according to claim 1, wherein the ginsenoside compound is a compound of Formula (A): 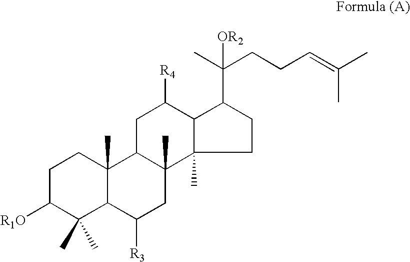
wherein R₁ is selected from the group consisting of H, acetyl, glucopyranosyl, glucopyranosyl-(2-1)-β-D-glucopyranosyl, glucopyranosyl-(2-1)-β-D-xylopyranosyl and glucopyranosyl-(2-1)-β-D-glucopyranosyl-(6-1)-xylopyranosyl; R₂ is selected from the group consisting of H, acetyl, glucopyranosyl, glucopyranosyl-(6-1)-β-D-glucopyranosyl, glucopyranosyl-(6-1)-β-D-xylopyranosyl, glucopyranosyl-(6-1)-α-L-arabinopyranosyl and glucopyranosyl-(6-1)-α-L-arabinofuranosyl; R₃ is selected from the group consisting of H, hydroxy, O-acetyl, O-β-D-glucopyranosyl, O-β-D-glucopyranosyl-(2-1)-β-D-glucopyranosyl, O-β-D-glucopyranosyl-(2-1)-β-D-xylopyranosyl and O-β-D-glucopyranosyl-(2-1)-α-L-rhamnopyranosyl; and R₄ is selected from the group consisting of H, hydroxyl and O-acetyl.

3. The method according to claim 1, wherein the ginsenoside compound is selected from the group consisting of:
ginsenoside Rb₁ of Formula I: 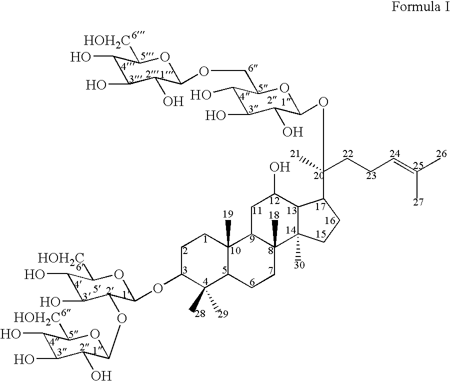
ginsenoside Rg₁ of Formula II: 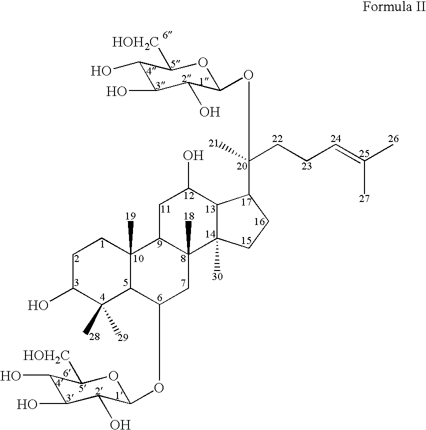
ginsenoside Rh₁ of Formula III: 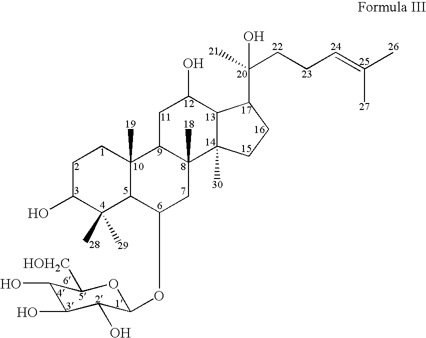
notoginsenoside R₁ of Formula V: 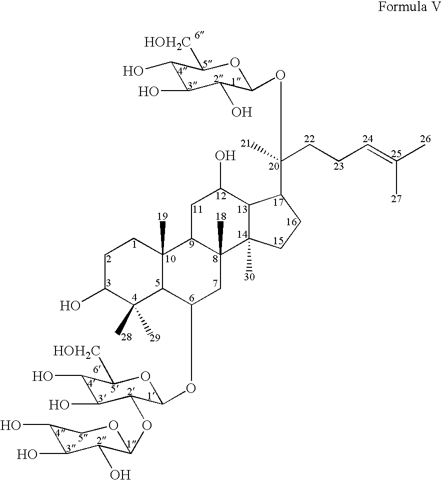
compound K of Formula VII: 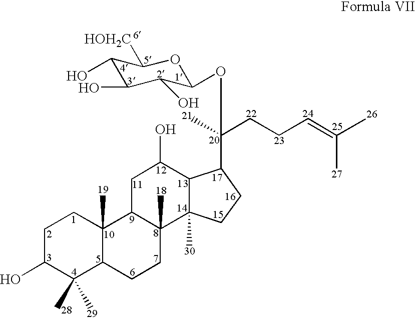
compound F₁ Formula VIII: 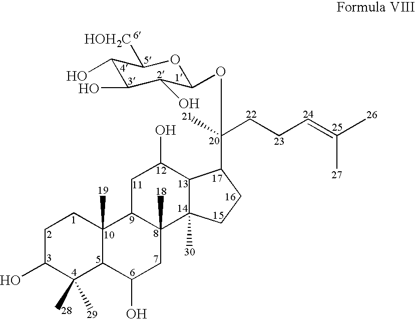
20(S)-protopanaxatriol of Formula IX:

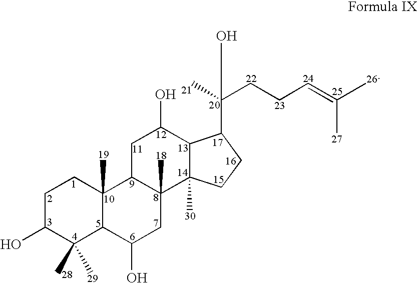

4. The method according to claim 1, wherein the nutrient is selected from the group consisting of glucose, an amino acid and a vitamin.

5. The method according to claim 4, wherein the amino acid is selected from the group consisting of arginine and tryptophan.

6. The method according to claim 4, wherein the vitamin is folate.

7. A method for enhancing the absorption of a nutrient in a subject, comprising the step of administering to the subject an effective amount of a ginsenoside compound purified from Panax notoginseng for facilitating transportation of the nutrient across gut cells of the subject.

8. The method according to claim 7, wherein the nutrient is selected from the group consisting of glucose, an amino acid and a vitamin.

9. The method according to claim 8, wherein the amino acid is selected from the group consisting of arginine and tryptophan.

10. The method according to claim 8, wherein the nutrient is glucose.

11. The method according to claim 10, wherein the absorption of glucose is enhanced by facilitating the transportation of glucose across the gut cells of the subject with administration of the ginsenoside compound at a concentration of about 0.001 μM to about 5 μM.

12. The method according to claim 10, wherein the ginsenoside compound is selected from the group consisting of ginsenoside Rb₁ of Formula I, compound K of Formula VII, ginsenoside F₁ of Formula VIII and 20(S)-protopanaxatriol of Formula IX.

13. The method according to claim 9, wherein the nutrient is arginine.

14. The method according to claim 12, wherein the absorption of arginine is enhanced by facilitating the transportation of arginine across the gut cells of the subject with administration of the ginsenoside compound at a concentration about 0.001 μM to about 5 μM.

15. The method according to claim 13, wherein the ginsenoside compound is selected from the group consisting of ginsenoside Rb₁ of Formula I, ginsenoside Rg₁ of Formula II, compound K of Formula VII, ginsenoside Rh₁ of Formula III, ginsenoside F₁ of Formula VIII and 20(S)-protopanaxatriol of Formula IX.

16. The method according to claim 9, wherein the nutrient is tryptophan.

17. The method according to claim 15, wherein the absorption of tryptophan is enhanced by facilitating transportation of tryptophan across the gut cells of the subject with administration of the ginsenoside compound at a concentration about 0.001 μM to about 5 μM.

18. The method according to claim 16, wherein the ginsenoside compound is selected from the group consisting of compound K of Formula VII and ginsenoside Rg₁ of Formula II.

19. The method according to claim 7, wherein the nutrient is folate.

20. The method according to claim 19, wherein the absorption of folate is enhanced by facilitating transportation of folate across the gut cells of the subject with administration of the ginsenoside compound at a concentration about 0.001 μM to about 5 μM.

21. The method according to claim 19, wherein the ginsenoside compound is selected from the group consisting of compound K of Formula VII and ginsenoside Rb₁ of Formula I.

22. A method for inhibiting the absorption of a nutrient in a subject, comprising the step of administering to the subject an effective amount of a ginsenoside compound purified from Panax notoginseng for moderating transportation of the nutrient across gut cells of the subject.

23. The method according to claim 22, wherein the nutrient is selected from the group consisting of glucose and a vitamin.

24. The method according to claim 23, wherein the nutrient is glucose.

25. The method according to claim 24, wherein the absorption of glucose is inhibited by moderating the transportation of glucose across the gut cells of the subject with administration of the ginsenoside compound at a concentration of about 0.001 μM to about 5 μM.

26. The method according to claim 24, wherein the ginsenoside compound is selected from the group consisting of ginsenoside Rg₁ of Formula II and ginsenoside Rh₁ of Formula III.

27. The method according to claim 22, wherein the nutrient is folate.

28. The method according to claim 27, wherein the absorption of folate is inhibited by moderating the transportation of folate across the gut cells of the subject with administration of the ginsenoside compound at a concentration of about 0.001 μM to about 5 μM.

29. The method according to claim 27, wherein the ginsenoside compound is selected from the group consisting of ginsenoside Rg₁ of Formula II and ginsenoside Rh₁ of Formula III.
